(19) United States
(12) Patent Application Publication
(10) Pub. No.: US 2019/0287666 A1
(43) Pub. Date: Sep. 19, 2019

(54) SYSTEMS AND METHODS FOR AUTOMATED PRESCRIPTION DISPENSING

(71) Applicants: Xiaoyan Qin, Wellesley, MA (US); Jeffrey Alan Miner Gruneich, Wellesley, MA (US)

(72) Inventors: Xiaoyan Qin, Wellesley, MA (US); Jeffrey Alan Miner Gruneich, Wellesley, MA (US)

(21) Appl. No.: 16/353,821

(22) Filed: Mar. 14, 2019

Related U.S. Application Data
(60) Provisional application No. 62/642,936, filed on Mar. 14, 2018.

(51) Int. Cl. G16H 20/13; G05B 19/4155

(52) U.S. Cl. CPC G16H 20/13; G05B 2219/45083; G05B 19/4155

(57) ABSTRACT

The presently described systems and methods for automated prescription dispensing provide a virtual prescription verification environment that enables a pharmacist to fill prescriptions, control and remotely verify the performance of pharmacy robotic devices, and counsel patients effectively through a highly secure application.

### RELATED APPLICATION

This application claims the benefit of, and priority to, U.S. Provisional Patent Application No. 62/642,936, filed Mar. 14, 2018, the contents of which are incorporated herein by reference in their entirety.

### BACKGROUND

The prescription drug industry includes several different organizations and professionals. These groups include the health care providers, pharmacies, insurance companies, federal agencies, state agencies, local agencies, and pharmaceutical firms. Each of these groups function, for the most part, separately from the others, and each has its own specific interests. The filling and dispensing of prescriptions is a daily occurrence. A pharmacist receives a prescription order and manually fills the order. The pharmacist verifies that the order has been filled correctly. The filled order can then be made available to a customer.

Retail prescription volume has been rising each year and the role of pharmacists in our current healthcare system is expanding rapidly too, including additional roles within hospitals and specialty practices, medication management, transitions of care, or new opportunities related to prescribing and administering medications.

### BRIEF DESCRIPTION OF THE DRAWINGS

To assist those of skill in the art in making and using a system for remotely dispensing prescriptions using a robotic device and associated methods, reference is made to the accompanying figures. The accompanying figures, which are incorporated in and constitute a part of this specification, illustrate one or more embodiments of the invention and, together with the description, help to explain the embodiments. The drawings are not necessarily to scale, or inclusive of all elements of a system, emphasis instead generally being placed upon illustrating the concepts, structures and techniques sought to be protected herein. Illustrative embodiments are shown by way of example in the accompanying drawings and should not be considered as limiting. In the figures:

FIG. 1 is a block diagram illustrating an automated prescription dispensing environment in accordance with illustrative embodiments.

FIG. 2 is a flowchart of a prescription dispensing method using a robotic device in accordance with an exemplary embodiment.

FIG. 3 is a block diagram showing a pharmacy computing device or a user computing device suitable for use in accordance with illustrative embodiments.

FIG. 4 depicts exemplary user interfaces in a virtual prescription verification environment suitable for use in accordance with illustrative embodiments.

### DETAILED DESCRIPTION

The description herein is presented to enable a person skilled in the art to create and use a system and method for automated prescription dispensing. Various modifications to the example embodiments are readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art can realize that the embodiments may be practiced without the use of these specific details. In other instances, well-known structures and processes are shown in block diagram form in order not to obscure the description of the embodiments with unnecessary detail. Thus, the present disclosure is not intended to be limited to the embodiments shown, but is to be accorded the widest scope consistent with the principles and features disclosed herein.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes multiple system elements, device components or method steps, those elements, components or steps can be replaced with a single element, component or step. Likewise, a single element, component or step can be replaced with multiple elements, components or steps that serve the same purpose. Moreover, while exemplary embodiments have been shown and described with references to particular embodiments thereof, those of ordinary skill in the art understand that various substitutions and alterations in form and detail can be made therein without departing from the scope of the invention. Further still, other aspects, functions and advantages are also within the scope of the invention.

Portions or all of the embodiments may be provided as one or more computer-readable programs or code embodied on or in one or more non-transitory mediums. The mediums may be, but are not limited to a hard disk, a compact disc, a digital versatile disc, a flash memory, a PROM, a RAM, a ROM, or a magnetic tape. In general, the computer-readable programs or code may be implemented in many computing languages.

Exemplary flowcharts are provided herein for illustrative purposes and are non-limiting examples of methods. One of ordinary skill in the art recognize that exemplary methods can include more or fewer steps than those illustrated in the exemplary flowcharts, and that the steps in the exemplary flowcharts can be performed in a different order than the order shown.

The prescription filling process can be labor intensive and is prone to human error either in the type of prescription dispensed or in the amount of doses for a given prescription. In the unfortunate event that a prescription is filled incorrectly, serious results can occur, including overdose or even death. Accordingly, care must be taken in the prescription filling process to make sure the prescription is accurately filled.

Some of the largest costs of operations of a pharmacy are the costs of inventory and the costs of labor. While electromechanical systems have been developed to address some of these deficiencies, several key deficiencies remain that are centered around a pharmacist's workflow. These deficiencies include the ability to remotely control and validate the performance of a robotic device during the filling of a prescription. Another deficiency is the inability to coordinate the splitting of pharmacist labor between tasks which can be done remotely and tasks which can be done locally in the pharmacy or adjacent to a medication dispensing machine. There is also a need to collaborate labor using one networked electronic system to manage the overall prescription filling process so that each medication may be dispensed at each step, safely and economically.

Embodiments described herein provide systems and methods for automated prescription dispensing. More particularly, embodiments enable a pharmacist to fill prescriptions, control and verify the performance of pharmacy automation robotic devices, and counsel patients effectively through a highly secure application over a network, remotely or in the pharmacy on their own device or a network-attached computer. As a result, pharmacists have more time to focus on better medication management, enhanced patient compliance, improved privacy and service for the patient, reduce costs of operations to the pharmacy, and a net improvement in overall patient care.

In one embodiment a system for automated filling and dispensing prescriptions includes a robotic device configured to fill and dispense prescriptions, an application providing a virtual prescription verification environment, and at least one camera or other imaging device that is situated to observe a robotic device filling a prescription. The system also includes a computing device equipped with one or more processors that is configured to execute the application providing the virtual prescription verification environment. The computing device is communicatively coupled to the robotic device and the camera. The virtual prescription verification environment receives a first input for a prescription for a patient, transmits instructions to the robotic device to fill the prescription based on the first input, receives visual data from the at least one imaging device of the robotic device filling the prescription, displays the visual data, receives a verification status of the filled prescription, and transmits an action instruction instructing the robotic device to perform an action associated with the filled prescription.

The prescription verification environment may also include a secure application that enables the pharmacist to communicate from the virtual prescription verification environment with at least one of a patient, a patient's physician, a patient's healthcare provider, and insurance payer. The pharmacist may interact with the computing device and the prescription verification environment when the pharmacist, the computing device, and a patient whose prescription is being filled are co-located or situated in different physical locations.

In another embodiment, a method for automated prescription dispensing includes receiving a first input, via a virtual prescription verification environment, for a prescription for a patient and transmitting instructions via the virtual prescription verification environment to the robotic device to fill the prescription based on the first input. The method also includes receiving visual data in the virtual prescription verification environment from at least one imaging device of the robotic device filling the prescription and displaying the visual data in the virtual prescription verification environment. The method further includes receiving in the virtual prescription verification environment an input verification status of a prescription based on the visual data, and transmitting an action instruction instructing the robotic device to perform an action associated with the filled prescription following the verification.

Other arrangements of embodiments of the invention that are disclosed herein include software programs to perform the method embodiment steps and operations summarized above and disclosed in detail below. More particularly, a non-transitory computer program product is one embodiment that has a computer-readable medium including computer program logic encoded thereon that when performed in a computerized device provides associated operations providing automated prescription dispensing as explained herein. The computer program logic, when executed on at least one processor with a computing system, causes the processor to perform the operations indicated herein as embodiments of the invention. Such arrangements of the embodiments are provided as software, code and/or other data structures arranged or encoded on a computer readable medium such as an optical medium (e.g., CD-ROM), floppy or hard disk or other a medium such as firmware or microcode in one or more ROM or RAM or PROM chips or as an Application Specific Integrated Circuit (ASIC) or as downloadable software images in one or more modules, shared libraries, etc. The software or firmware or other such configurations can be installed onto a computerized device to cause one or more processors in the computerized device to perform the techniques explained herein as embodiments. Software processes that operate in a collection of computerized devices, such as in a group of data communications devices or other entities can also provide the system described in the embodiments. The system can be distributed between many software processes on several data communications devices, or all processes could run on a small set of dedicated computers, or on one computer alone.

It is to be understood that the embodiments can be embodied strictly as a software program, as software and hardware, or as hardware and/or circuitry alone. Note that each of the different features, techniques, configurations, etc. discussed in this disclosure can be executed independently or in combination. Accordingly, the present embodiments can be embodied and viewed in many different ways. Also, note that this summary section herein does not specify every embodiment and/or incrementally novel aspect of the present disclosure or claimed embodiments. Instead, this summary only provides a preliminary discussion of different embodiments and corresponding points of novelty over conventional techniques. For additional details, elements, and/or possible perspectives (permutations) of the embodiments, the reader is directed to the Detailed Description section and corresponding figures of the present disclosure as further discussed below.

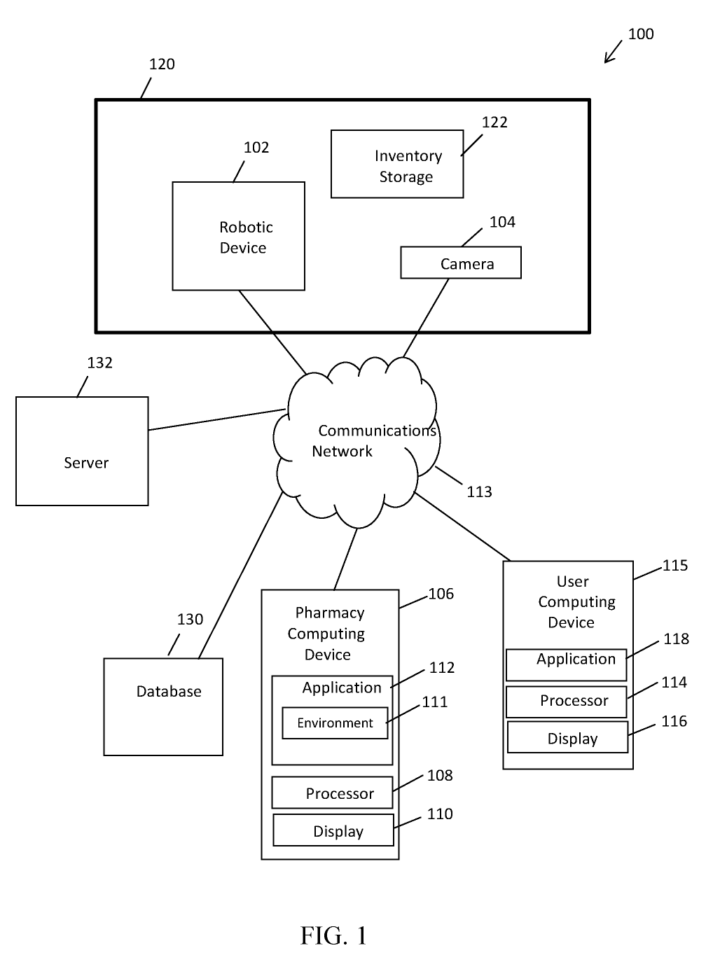

Described in detail herein are methods and systems for remotely dispensing prescriptions using a robotic device. Referring to FIG. 1, an exemplary network environment suitable for a system 100 for remotely dispensing prescriptions using a robotic device 102 is illustrated. System 100 includes a robotic device 102, at least one imaging device such as camera 104, a pharmacy computing device 106, an inventory storage 122, a database 130, and a central server 132. System 100 may also include a user computing device 108. The robotic device 102 has access to inventory storage 122 where prescriptions materials are stored before being used to fill prescriptions. The camera 104 or other imaging device is disposed so as to include the robotic device within its image capturing field when the robotic device attempts to fill prescriptions. The robotic device 102 and the camera 104 may be located in a pharmacy or similar setting 120. Alternatively, the robotic device 102 may be located remotely from the pharmacy when the inventory storage 122 is offsite. A communication network 113 may be used for communications between the pharmacy computing device 106, the user computing device 108, the robotic device 102, and the camera 104.

In an exemplary embodiment, the pharmacy computing device 106 and/or the user computing device 108 may be a laptop computer, a desktop computer, a smartphone, a tablet, or other mobile computing device. The pharmacy computing device 106 includes one or more processors 108 and a display 110. The processor 108 is configured to execute the application 112. Application 112 provides a virtual prescription verification environment 111 that includes a user interface for accepting input and displaying data. The virtual prescription verification environment 111 may also include a secure application 112 that provides a secures means of communicating from within the virtual prescription verification environment 111 to other users accessing other computing devices. For example, a corresponding secure application 118 may be executed on user computing device 108 to allow a user/patient to communicate with the pharmacist via the virtual prescription verification environment 111. As a non-limiting example, the robotic device 102 and camera 104 may be located within a physical facility 120 such as a pharmacy distribution center or pharmacy retail store. In one embodiment, the camera 104 is located on the robotic device 102. In another embodiment, the camera 102 is located proximate the robotic device 102.

The virtual prescription verification environment 111 may include a user interface enabling the individual (e.g., the pharmacist) operating the pharmacy computing device 106 to view the camera feed. The virtual prescription verification environment 111 is used to control the transmission of instructions to the robotic device 102 and verify the prescription filling operations performed by the robotic device as further explained herein. Also shown is a database 130 comprising prescription information for prescriptions and patients. It will be appreciated that database 130 may be local or remote to the pharmacy or pharmacist's location.

The user computing device 115 includes a processor 114 and a display 116. The processor 114 is configured to execute a secure application 118 installed on the user computing device 115. The application 118 may also generate a user interface enabling a user (e.g., the patient) operating the user computing device 115 to, among other things, consult with a pharmacist.

In an embodiment, the virtual prescription verification environment 111 provided by application 112 receives a first input that includes identification of medication and patient information for an electronic prescription . The electronic prescription information is available to the pharmacist to check. The first input may be received in a number of ways by the virtual prescription verification environment 111. The pharmacist or patient may enter the information via a user interface provided in the virtual prescription verification environment 111 on the pharmacy computing device 106. Alternatively, the first input may be received from database 130 or from another remote location communicating with the virtual prescription verification environment 111. Based on the receipt of the first input, the virtual prescription verification environment 111 transmits the prescription details to an automatic prescription dispensing robotic device 102 at a desired location. The robotic device 102 performs necessary operations to fill, and optionally label the bottle with patient information and drug information and count the pills for the pharmacist. The camera 104 or other imaging device records the robotic device's filling operations and provides visual data to the virtual prescription verification environment 111 to enable the pharmacist to do the final quality verification. The visual data and associated information from database 130 and/or the first input appears on the pharmacist's pharmacy computing device 106 in the virtual prescription verification environment 111 including label information, drug information, and/or an image of the drug in the bottle via the camera 104. In some embodiments, information regarding a copayment may be sent to the patient's user computing device 115, along with a request for the patient to confirm a pick up location and to pay the copayment via their application 118.

After the virtual prescription verification environment 111 has received a verification status for the filling operation, an action instruction for an action is transmitted to the robotic device 102. For example, the action may transmit instructions to the robotic device 102 to cap the bottle of the filled prescription and transfer the filled prescription to the automatic transaction machine (ATM), and wait for the patient to pick it up at the ATM window. If the pharmacist or system found any error on the prescription, she or he can stop the robotic device 102 from dispensing the prescription. The pharmacist or system can also direct the robotic device 102 to remake another bottle. The robot 102 sends confirmation information to a central server 132 where the confirmation can be made available to a patient's user computing device 115, inform the patient the prescription is ready for pick up, and optionally also inform the patient's physician or other recipients.

In one embodiment, as the patient arrives at the pharmacy ATM location, the patient can complete the transaction in a fully automated manner without interacting with anyone. The patient may scan their code, card, or security key on the user computing device 115, and a security scan allows the ATM to dispense the prescription.

In one embodiment, the ability to counsel patients quickly and privately for the convenience of both the patient and the pharmacist may take place in a remote, non-pharmacy setting over the network 113 on their own networked computerized devices (e.g., the pharmacy computing device 106 and the user computing device 115). Application 112 and application 118 may be used on the pharmacy computing device 106 and the user computing device 115, respectively, which allows a patient and a pharmacist to communicate privately, helps manage a cue of waiting patients, and integrates with remote computerized records so that the pharmacist can quickly understand the entire picture for the patient including a range of artificial intelligence-generated and ranked questions the pharmacist might verify to provide a more efficient patient consult. The patient may consult with a pharmacist at that store or at another remote location thru the application 118 on the user computing device 115. The patient can request or deny the counsel offer through the application 118. If patient accepts the counsel offer, a live pharmacist appears on the application 118. The application 112 installed on the pharmacy computing device 106 enables the pharmacist to use the pharmacy computing device 106 to communicate with the patient in real time via the application 118.

In an alternative embodiment, the application 112 receives an identification of medication and patient information for an electronic prescription from remote database 130. The electronic prescription information is available to the pharmacist to check. The patient may confirm an address for mail order or delivery service using the application 118 on the patient's user computing device 115. In some embodiments, the user can also pay a copayment on the application 118. Then the prescription details are sent to an automatic prescription dispensing robotic device 102 at a pharmacy distribution center or pharmacy retail store to fill and optionally label the bottle with patient information and drug information, optionally count the pills for the pharmacist. All the corresponding information appears on the pharmacist's pharmacy computing device 106, including label information, drug information, and an image of the drug in the bottle via a live camera.

After the pharmacist or an automated process checks the final product, he or she allows the robotic device 102 to cap the bottle of the filled prescription whereupon the filled prescription is ready to be transported to the patient via mail order or delivery service with the robotic device transporting the prescription to a location to start that delivery. If the pharmacist found any error on the prescription, she or he can stop the robotic device 102 from dispensing the prescription. The pharmacist can also direct the robotic device 102 to remake another bottle. The robotic device 102 may send confirmation information indicating a successful filling of the prescription to a central server 132 where the confirmation can be made available to a patient's user computing device 108, inform the patient the prescription is filled, in transit, or has arrived and optionally also inform the patient's physician or other recipients.

As the patient receives the prescription, the patient can scan a code on the package or on the prescription label to verify receipt and request or deny a consultation with a pharmacist remotely thru the application 118 on their user computing device 115. If patient accepts the counsel offer, a live pharmacist appears on the application 118. The virtual prescription verification environment 111 provided by application 112 installed on the pharmacy computing device 106 enables the pharmacist to use the pharmacy computing device 106 to communicate with the patient in real time via the application 118.

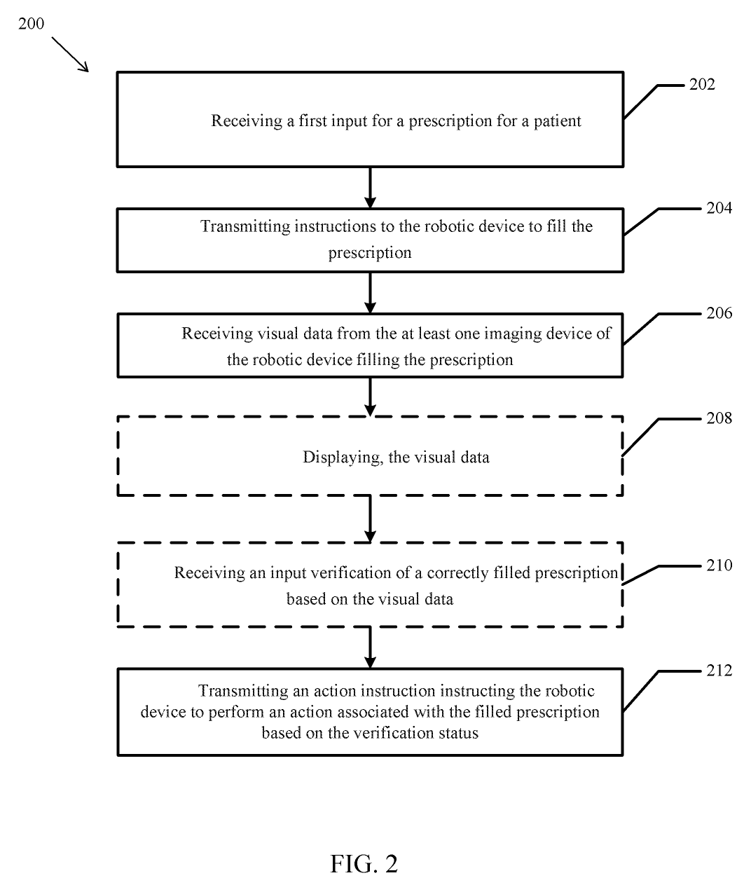

FIG. 2 is an example flowchart showing illustrative processing that can be implemented for remotely dispensing prescriptions using a robotic device in accordance with illustrative embodiments. The sequence begins with the virtual prescription verification environment receiving a first input for a prescription to be filled for a patient (step 202). Based on the first input, the virtual prescription verification environment is used to transmit instructions to the robotic device to fill the prescription (step 204). The virtual prescription verification environment receives visual data from at least one imaging device that is disposed to watch the filling operations performed by the robotic device (step 206). In one embodiment, the visual data is displayed in the virtual prescription verification environment (step 208) along with corresponding information related to the prescription so that the pharmacist can enter a verification status indicating whether the prescription was properly filled (step 210). In an alternate embodiment, the visual data is examined by a computer vision process or other image processing to determine whether the prescription has been properly filled and based on that examination a verification status is programmatically generated. Following the determination of the verification status, the virtual prescription verification environment transmits an action instruction based on the verification status to the robotic device instructing the robotic device to perform an action associated with the filled prescription (step 212). In one embodiment, the action instruction is programmatically generated. In another embodiment, the action instruction results from additional input entered by the pharmacist.

In one embodiment, the pharmacist, the computing device providing the virtual prescription verification environment, and the patient are co-located. In another embodiment, the pharmacist, the computing device providing the virtual prescription verification environment, and the patient are situated in multiple different physical locations. In one embodiment, the pharmacist is compensated on a per prescription basis.

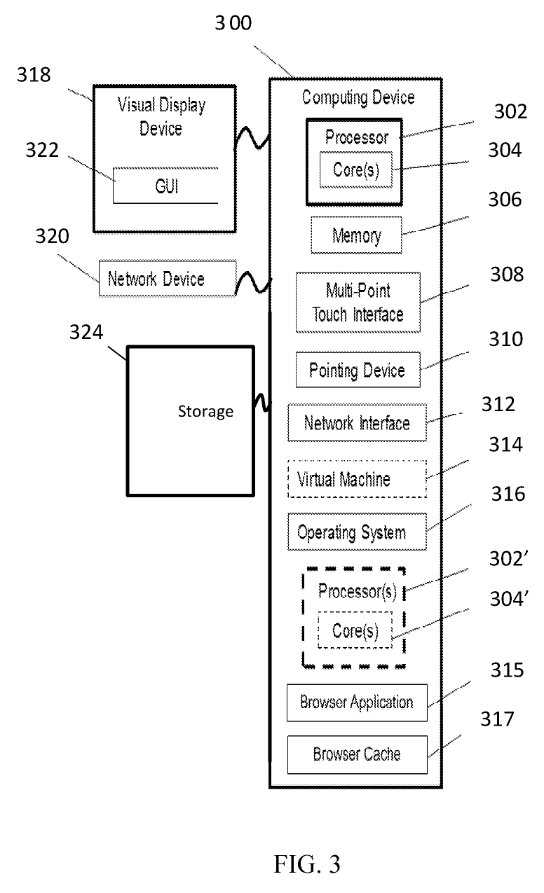

FIG. 3 is a block diagram showing a pharmacy computing device or a user computing device suitable for use in accordance with illustrative embodiments. In an exemplary embodiment, computing device 300 is a pharmacy computing device 106 or a user computing device 115 shown in FIG. 1. Computing device 300 includes one or more non-transitory computer-readable media for storing one or more computer-executable instructions or software for implementing exemplary embodiments described herein. The non-transitory computer-readable media can include, but are not limited to, one or more types of hardware memory, non-transitory tangible media (for example, one or more magnetic storage disks, one or more optical disks, one or more USB flash drives), and the like. For example, a memory 306 included in computing device 300 can store computer-readable and computer-executable instructions or software for implementing exemplary embodiments described herein. Computing device 300 also includes a processor 302 and an associated core 304, and optionally, one or more additional processor(s) 302′ and associated core(s) 304′ (for example, in the case of computer systems having multiple processors/cores), for executing computer-readable and computer-executable instructions or software stored in memory 306 and other programs for controlling system hardware. Processor 302 and processor(s) 302′ can each be a single core processor or multiple core (304 and 304′) processor. Computing device 300 may also include a browser application 315 and a browser cache 317 to enable a user to access information on computing device 300.

Virtualization can be employed in computing device 300 so that infrastructure and resources in the computing device can be shared dynamically. A virtual machine 314 can be provided to handle a process running on multiple processors so that the process appears to be using only one computing resource rather than multiple computing resources. Multiple virtual machines can also be used with one processor.

Memory 306 can include a computer system memory or random access memory, such as DRAM, SRAM, EDO RAM, and the like. Memory 306 can include other types of memory as well, or combinations thereof. In some embodiments, a customer can interact with computing device 300 through a graphical user interface (GUI) 322 associated with a visual display device 318, such as a touch screen display or computer monitor. Visual display device 318 may also display other aspects, elements and/or information or data associated with exemplary embodiments. Computing device 300 may include other I/O devices for receiving input from a customer, for example, a keyboard or any suitable multi-point touch interface 308, a pointing device 310 (e.g., a pen, stylus, mouse, or trackpad). The keyboard 308 and pointing device 310 may be coupled to visual display device 318. Computing device 300 may include other suitable conventional I/O peripherals.

Computing device 300 can also include one or more storage devices 324, such as a hard-drive, CD-ROM, or other computer readable media, for storing data and computer-readable instructions and/or software, that implements embodiments of the system, as described herein, or portions thereof. Exemplary storage device 324 can also store one or more storage devices for storing any suitable information required to implement exemplary embodiments.

Computing device 300 can include a network interface 312 configured to interface via one or more network devices 320 with one or more networks, for example, Local Area Network (LAN), Wide Area Network (WAN) or the Internet through a variety of connections including, but not limited to, standard telephone lines, LAN or WAN links (for example, 802.11, T1, T3, 56kb, X.25), broadband connections (for example, ISDN, Frame Relay, ATM), wireless connections, controller area network (CAN), or some combination of any or all of the above. The network interface 312 can include a built-in network adapter, network interface card, PCMCIA network card, card bus network adapter, wireless network adapter, USB network adapter, modem or any other device suitable for interfacing computing device 300 to any type of network capable of communication and performing the operations described herein. Moreover, computing device 300 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

Computing device 300 can run any operating system 316, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 316 can be run in native mode or emulated mode. In an exemplary embodiment, the operating system 316 can be run on one or more cloud machine instances.

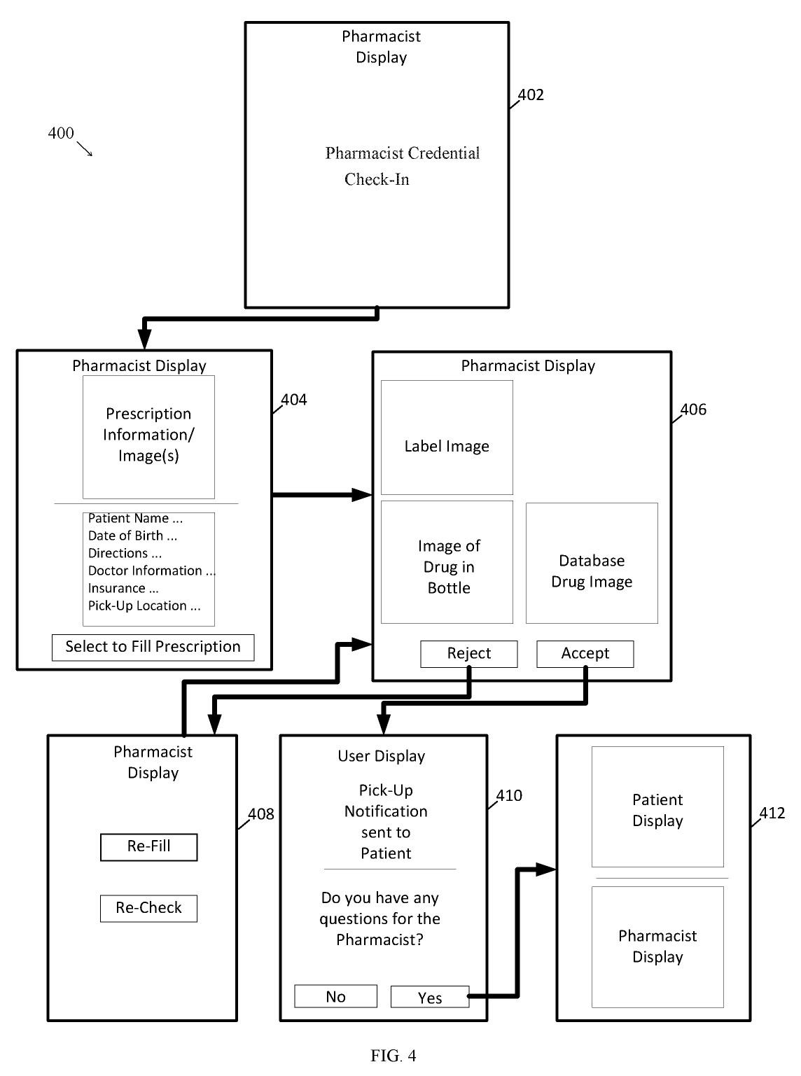

FIG. 4 illustrates a flowchart 400 of exemplary user interfaces suitable for use in accordance with illustrative embodiments. Pharmacist user interfaces 402-408 are shown within a virtual prescription verification environment provided by an executing application. Pharmacist user interface 402 is configured to receive credential/authentication information (e.g., a username and password) for a pharmacist to log into the virtual prescription verification environment. Pharmacist user interface 404 is configured to display prescription information for a patient (e.g., a name of a prescription, quantity prescribed, etc.) and/or an image(s) of the prescription (e.g., an image of the written prescription, a photo of the prescribed medication) for the patient and the patient's biographical information (e.g., name of patient, date of birth of the patient, primary care physician of the patient, preferred prescription pick-up location, etc.). The prescription data may have been received by the system in a number of ways. As non-limiting examples, the prescription information may be manually entered into the system by a pharmacist following receipt of a paper prescription order, may have been transmitted electronically to the application providing the virtual prescription verification environment or may have been retrieved from a database of prescription information, Using pharmacist user interface 404, the pharmacist can make a selection to fill the prescription, at which point the virtual prescription verification environment transmits instructions to the robotic device to fill the prescription. The virtual prescription verification environment receives visual data from at least one imaging device of the robotic device filling the prescription. Using pharmacist user interface 406, the virtual prescription verification environment displays the visual data, such as, but not limited to, an image of a label on the prescription bottle and an image of the medication in the filled prescription bottle. In some embodiments, pharmacist user interface 406 may further display an image of a different filled prescription bottle of the medication for comparison purposes. In some embodiments, the visual data displayed in the virtual prescription verification environment may include a video feed allowing the pharmacist to observe some or all of the filling process performed by the robot. The pharmacist may then use pharmacist user interface 406 to provide a verification status. The pharmacist may provide a valid verification status by making a selection authorizing the filled prescription or may provide an invalid verification status by making a selection to reject the filled prescription.

Based on the verification status, an action instruction is transmitted to the robotic device instructing the robotic device to take a further action with respect to the prescription. If the pharmacist verified the filling operation, the action instruction may instruct the robotic device to deliver the filled prescription to a designated area for eventual delivery to/pickup by the patient. If the pharmacist rejects the filled prescription, the pharmacist is directed to pharmacist user interface 408 where the pharmacist can make a selection via the UI to transmit an action instruction that directs the robotic device to re-fill another prescription or make a selection via the UI to re-check the visual data. The pharmacist is then redirected to the pharmacist user interface 406.

If the pharmacist has accepted the filled prescription, confirmation that the prescription is ready for pick-up is transmitted to the patient and displayed on a patient user interface 410 shown on the patient's user computing device, while optionally also informing the patient's physician or other designated recipients. Using patient user interface 410, the patient is also able to select an option to speak with the pharmacist via the patient's user computing device via a secure link.

If the patient selects the option to speak with the pharmacist, both the pharmacist and the patient are directed to user interface 412, which displays a real-time video feed of the patient and the pharmacist.

One of ordinary skill in the art recognizes that the interfaces illustrated above can include more, fewer, or different features than those illustrated in the exemplary illustrations, and that the steps in the exemplary flowchart of FIG. 2 can be performed in a different order than the order shown.

Having described certain embodiments, which serve to illustrate various concepts, structures, and techniques sought to be protected herein, it is apparent to those of ordinary skill in the art that other embodiments incorporating these concepts, structures, and techniques may be used. Elements of different embodiments described hereinabove may be combined to form other embodiments not specifically set forth above and, further, elements described in the context of a single embodiment may be provided separately or in any suitable sub-combination. Accordingly, it is submitted that the scope of protection sought herein should not be limited to the described embodiments but rather should be limited only by the spirit and scope of the following claims.

We claim:

1. A system for automated prescription dispensing, comprising:
a robotic device configured to fill and dispense prescriptions;
an application that when executed provides a virtual prescription verification environment;
at least one imaging device situated to observe the robotic device filling a prescription; and
a computing device equipped with a processor and configured to execute the application providing the virtual prescription verification environment, the computing device communicatively coupled to the robotic device and the imaging device, the virtual prescription verification environment when executed:
receives a first input for a prescription for a patient;
transmits instructions to the robotic device to fill the prescription based on the first input;
receives visual data from the at least one imaging device of the robotic device filling the prescription;
displays the visual data;
receives an input verification status of a prescription based on the visual data; and
transmits an action instruction instructing the robotic device to perform an action associated with the filled prescription based on the verification status.

2. The system of claim 1, wherein the input verification status is valid and the action is dispensing the filled prescription.

3. The system of claim 1, wherein the input verification status is invalid and the action is an instruction to the robotic device to refrain from dispensing the filled prescription or reject the filled prescription.

4. The system of claim 1, further comprising:
a database including prescription information for a plurality of prescriptions, wherein the virtual prescription verification environment:
obtains prescription information for the filled prescription from the database; and
displays the prescription information for the filled prescription.

5. The system of claim 1, further comprising:
a database including patient information for a plurality of patients, wherein the virtual prescription verification environment:
obtains patient information for a specified patient from the database; and
displays the patient information.

6. The system of claim 1, wherein the virtual prescription verification environment includes a secure application that enables the pharmacist to communicate via the secure application with at least one of the patient, a patient's physician, a patient's healthcare provider, and a patient's insurance payer.

7. The system of claim 1, wherein the visual data includes images of at least one of a label on a prescription bottle associated with the filled prescription and contents of the prescription bottle associated with the filled prescription.

8. The system of claim 1, further comprising at least one computer vision algorithm verifying an accuracy of the filled prescription.

9. The system of claim 1, wherein a pharmacist interacts with the computing device and the prescription verification environment, wherein the pharmacist, the computing device, and a patient whose prescription is being filled are co-located.

10. The system of claim 1, wherein the pharmacist interacts with the computing device and the prescription verification environment, and wherein the pharmacist and the patient are situated in different physical locations.

11. A computing device-implemented method for automated prescription dispensing, comprising:
receiving a first input, via a virtual prescription verification environment, for a prescription for a patient;
transmitting instructions via the virtual prescription verification environment to the robotic device to fill the prescription based on the first input;
receiving visual data in the virtual prescription verification environment from at least one imaging device of the robotic device filling the prescription;
displaying, the visual data in the virtual prescription verification environment;
receiving in the virtual prescription verification environment an input verification status of a prescription based on the visual data; and
transmitting an action instruction from the virtual prescription verification environment to the robotic device instructing the robotic device to perform an action associated with the filled prescription based on the verification status.

12. The method of claim 11, wherein the input verification status is valid and the action is dispensing the filled prescription.

13. The method of claim 11, wherein the input verification status is invalid and the action is an instruction to the robotic device to refrain from dispensing the filled prescription, or reject the filled prescription.

14. The method of claim 11, further comprising:
obtaining prescription information for the filled prescription from a database that includes prescription information for a plurality of prescriptions; and
displaying the prescription information for the filled prescription.

15. The method of claim 11, further comprising:
obtaining patient information for a specified patient from a database that includes patient information for a plurality of patients; and
displaying the specified patient information.

16. The method of claim 11, further comprising:
communicating via a secure application in the virtual prescription verification environment with at least one of the patient, a patient's physician, and a patient's healthcare provider, and a patient's insurance payer.

17. The method of claim 11, wherein the visual data includes images of at least one of a label on a prescription bottle associated with the filled prescription and contents of the prescription bottle associated with the filled prescription.

18. The method of claim 11, further comprising at least one computer vision algorithm verifying an accuracy of the filled prescription.

19. A non-transitory computer readable medium storing instructions executable by a computing device equipped with one or more processors, wherein execution of the instructions causes at least one computing device to:
receive a first input, via a virtual prescription verification environment, for a prescription for a patient;
transmit instructions via the virtual prescription verification environment to the robotic device to fill the prescription based on the first input;
receive visual data in the virtual prescription verification environment from at least one imaging device of the robotic device filling the prescription;
display, the visual data in the virtual prescription verification environment;
receive in the virtual prescription verification environment an input verification status of a prescription based on the visual data; and
transmit an action instruction from the virtual prescription verification environment to the robotic device instructing the robotic device to perform an action associated with the filled prescription based on the verification status.

20. The medium of claim 19, wherein the verification status is valid and the instructions when executed further cause the at least one computing device to:
transmit instructions to the robotic device to dispense the filled prescription.
